(19) United States
(12) Patent Application Publication
(10) Pub. No.: US 2026/0106028 A1
(43) Pub. Date: Apr. 16, 2026

(54) Systems and Methods of Supplementing Data Sets with Virtual Data for Medical Diagnoses

(71) Applicant: University of Virginia Patent Foundation, Charlottesville, VA (US)

(72) Inventors: Benjamin J. Lobo, Charlottesville, VA (US); Mohammadreza Ganjiarjenaki, Charlottesville, VA (US); Chiara Fabris, Charlottesville, VA (US); Boris P Kovatchev, Charlottesville, VA (US)

(21) Appl. No.: 19/358,415

(22) Filed: Oct. 14, 2025

Related U.S. Application Data
(60) Provisional application No. 63/706,550, filed on Oct. 11, 2024.

(51) Int. Cl. G16H 50/20; G16H 50/30; G16H 50/70; G16H 70/60

(52) U.S. Cl. CPC G16H 50/20; G16H 50/30; G16H 50/70; G16H 70/60

(57) ABSTRACT

Blood glucose control data is used as a medical diagnostic tool with a computer storing software identifies selected segments of archival blood glucose traces within the data set and estimates corresponding values of glycated hemoglobin for the selected segments of archival blood glucose traces. The corresponding values of glycated hemoglobin are stored in the computer memory. Using the corresponding values of glycated hemoglobin and the method associates respective data set profiles of respective subjects, from across the data set, with one of the selected segments of archival blood glucose traces. Adding at least one virtual continuous glucose monitoring (CGM) trace to each of the selected segments of blood glucose traces across the data set allows for calculating time in range (TIR) data for the data set profiles and identifying correlations between the TIR data and occurrences of physical maladies with the data set.

### CROSS-REFERENCE TO RELATED APPLICATIONS

This application claims priority to and the benefit of U.S. provisional patent Application No. 63/706,550, filed on Oct. 11, 2024, and entitled The Virtual DCCT: Adding Continuous Glucose Monitoring to a Landmark Clinical Trial for Prediction of Microvascular Complications, the disclosure of which is hereby incorporated by reference herein in its entirety.

### STATEMENT OF GOVERNMENT RIGHTS None.

### BACKGROUND

Glycated hemoglobin (HbA1c) has been the gold standard for assessing the risk of diabetic complications since the results of the landmark Diabetes Control and Complications Trial (DCCT) were published in 1993.1 The DCCT demonstrated that intensive insulin treatment reduces microvascular complications in type 1 diabetes, and established that glycated hemoglobin levels predict the rates of retinopathy, nephropathy, and neuropathy in this population.1 Despite the unequivocal value of glycated hemoglobin in assessing glycemic control, certain limitations have been identified. In particular, there is considerable interindividual variability in the relationship between glycated hemoglobin and the mean glucose concentration, with about 50% of people with diabetes having a laboratory-measured glycated hemoglobin level that is at least 0.3 percentage points higher or lower than would be predicted from their mean glucose level and 25% having discordance of 0.5 percentage points or greater.2 This discordance has been attributed to interindividual differences in red blood cell lifespan.3 Additionally, racial differences exist in the glycated hemoglobin-mean glucose relationship, such that for a given mean glucose, glycated hemoglobin on average is about 0.4 percentage points higher in African Americans than in Whites.4 Use of continuous glucose monitoring (CGM) is now part of standard care for people with type 1 diabetes and insulin treated type 2 diabetes, and CGM metrics have become a strong complement to glycated hemoglobin in diabetes management.5 The percentage of glucose values between 70 and 180 mg/dL, referred to as time-in-range (TIR), has become a key measure of glucose control.6-8 TIR is largely a measure of hyperglycemia and as such, is correlated with glycated hemoglobin.9 Fabris et al. demonstrated that when hemoglobin glycation was modeled by a first-order differential equation driven by TIR, the correlation between TIR and glycated hemoglobin increased from about 0.70 to >0.90.10 However, the Food and Drug Administration (FDA) has not accepted CGM metrics as endpoints for making efficacy claims in clinical trials conducted for the approval of a new drug or device, indicating that TIR is a biomarker that has not been established as a surrogate for a clinical outcome.11 Thus, there is a need to demonstrate that TIR is associated with important clinical outcomes, similar to the demonstration in the DCCT of the association of glycated hemoglobin with microvascular complication rates.

This disclosure utilizes raw data that is amenable to numerous kinds of mathematical and computer implemented methods of analysis. In some embodiments, artificial intelligence and machine learning techniques may be used in optional embodiments of this disclosure. Machine Learning (ML) and Artificial Intelligence (AI) systems are in widespread use in customer service, marketing, and other industries, including medicine and science. Machine learning is considered a subset of more general artificial intelligence operations, and AI endeavors may utilize numerous instances of machine learning to make decisions, predict outputs, and perform human-like intelligent operations. Machine learning protocols typically involve programming a model that instantiates an appropriate algorithm for a given computing environment and training the model on a particular data set or domain with known historical results. The results are generally known outputs of many combinations of parameter values that the algorithm accesses during training. The model uses numerous statistical and mathematical operations to learn how to make logical decisions and generate new outputs based on the historical training data. Machine learning (ML) includes, but is not limited to, a number of models such as neural networks, deep learning algorithms, support vector machines, data clustering, regression models, and Monte Carlo simulations. Other models may utilize linear regression, logistic regression, support vector machines, K-means clustering, classification models such as a binary classifier or a multi-class classifier, clustering models, anomaly detection, other supervised learning models, and even combinations of one or more machine language model types. Most of these take vectors of data as inputs.

The term “artificial intelligence,” therefore, includes any technique that enables one or more computing devices or comping systems (i.e., a machine) to mimic human intelligence. Artificial intelligence (AI) includes, but is not limited to, knowledge bases, machine learning, representation learning, and deep learning. The term “machine learning” is generally a subset of AI that enables a machine to acquire knowledge by extracting patterns from raw data.

The term “representation learning” may be used as a subset of machine learning that enables a machine to automatically discover representations needed for feature detection, prediction, or classification from raw data. Representation learning techniques include, but are not limited to, autoencoders.

The term “deep learning” may also be considered a subset of machine learning that enables a machine to automatically discover representations needed for feature detection, prediction, classification, etc. using layers of processing. Deep learning techniques include, but are not limited to, artificial neural network or multilayer perceptron (MLP).

Machine learning models include supervised, semi-supervised, and unsupervised learning models. In a supervised learning model, the model learns a function that maps an input (also known as feature or features) to an output (also known as target or target) during training with a labeled data set (or dataset). In an unsupervised learning model, the model learns a function that maps an input (also known as feature or features) to an output (also known as target or target) during training with an unlabeled data set. In a semi-supervised model, the model learns a function that maps an input (also known as feature or features) to an output (also known as target or target) during training with both labeled and unlabeled data.

Some machine learning models are designed for a specific data set or domain and are highly expert at handling the nuances within that narrow domain. It is with respect to these and other considerations that the various aspects of the present disclosure as described below are presented.

This disclosure combines algorithms deciphered by artificial intelligence and machine learning with currently known systems and models that gather data from a patient on a real time basis. Accordingly, this disclosure can utilize sensors and medical equipment that improve a system's ability to diagnose and treat a patient.

Brackets with numerals therein refer to references cited the below disclosure.

### SUMMARY

Blood glucose control data is used as a medical diagnostic tool with a computer storing software identifies selected segments of archival blood glucose traces within the data set and estimates corresponding values of glycated hemoglobin for the selected segments of archival blood glucose traces. The corresponding values of glycated hemoglobin are stored in the computer memory. Using the corresponding values of glycated hemoglobin and the method associates respective data set profiles of respective subjects, from across the data set, with one of the selected segments of archival blood glucose traces. Adding at least one virtual continuous glucose monitoring (CGM) trace to each of the selected segments of blood glucose traces across the data set allows for calculating time in range (TIR) data for the data set profiles and identifying correlations between the TIR data and occurrences of physical maladies with the data set.

### BRIEF DESCRIPTION OF THE DRAWINGS

Reference will now be made to the accompanying drawings, which are not necessarily drawn to scale.

FIG. 1. is an illustration of timing of collection of glycated hemoglobin and capillary blood glucose data in two participants from the intensive treatment (Patient ID: 45) and conventional treatment group (Patient ID: 151). Triangles represent 7-point capillary glucose profiles, typically taken quarterly; circles represent glycated hemoglobin determinations taken monthly in the intensive treatment group and quarterly in the conventional treatment group.

FIG. 2. shows time-in-range (TIR, 70-180 mg/dL) by treatment group over time: TIR was calculated for each participant and quarter based on 14 days of virtual CGM data. The points represent medians of the quarterly values, and the lines represent 25th and 75th percentiles of the yearly values. TIR of 50% was a clear demarcation line between the experimental and control groups, with most participants in the intensive group having TIR above 60% and most participants in the conventional group having TIR below 40%. CGM, continuous glucose monitoring.

FIG. 3. shows rate of retinopathy by time-in-range (TIR, 70-180 mg/dL: TIR was calculated for each participant at each 6-month visit based on 14 days of virtual CGM data. The figure includes data from both treatment groups. Points represent the crude rate of retinopathy within deciles of TIR. Lines represent the estimate and 95% confidence interval from a Poisson regression model for rate of retinopathy as a function of log TIR.

FIG. 4. shows rate of microalbuminuria by time-in-range (TIR, 70-180 mg/dL). TIR was calculated for each participant at each 6-month visit based on 14 days of virtual CGM data. The below includes data from both treatment groups. Points represent the crude rate of microalbuminuria within deciles of TIR. Lines represent the estimate and 95% confidence interval from a Poisson regression model for rate of microalbuminuria as a function of log TIR.

FIG. 5 is a Supplementary Figure (S1) for Glycated Hemoglobin by Treatment Group over Time. FIG. 5A is from the primary DCCT paper1. FIG. 5B is the same results reproduced thirty years later. In both panels, the points represent medians of the quarterly values, and the lines represent 25th and 75th percentiles of the yearly values.

FIG. 6 is a Supplementary Figure (S2) for Rate of Retinopathy by Mean Glycated Hemoglobin. FIG. 6A is from the primary DCCT paper1. This figure is based on data from the intensive group only. FIG. 6B is the same analysis reproduced for both treatment arms combined (intensive and conventional), showing that a very similar relationship is valid for the entire DCCT population regardless of arm assignment. In both panels, points represent the crude rate of retinopathy within deciles of mean glycated hemoglobin. Lines represent the estimate and 95% confidence interval from a Poisson regression model for rate of retinopathy as a function of log mean glycated hemoglobin.

FIG. 7 is a Supplementary Figure (S3) showing Sensitivity of Rate of Retinopathy by Time in Range 70-180 mg/dL to Number of Days of CGM Data. In the figures, points represent the crude rate of retinopathy within deciles of time in range 70-180 mg/dL. Lines represent the estimate and 95% confidence interval from a Poisson regression model for rate of retinopathy as a function of log time in range 70-180 mg/dL.

FIG. 8 is a Supplementary Figure (S4) showing Boxplots of Glycated Hemoglobin and Time in Range 70-180 mg/dL by Development of Neuropathy at 5 years. FIG. 8A shows boxplots of mean glycated hemoglobin by diagnosis of neuropathy at 5 years among participants who did not have neuropathy at baseline. Mean glycated hemoglobin was calculated for each participant by taking a weighted average of all measurements on and prior to the 5-year visit. In each box, the black dot represents the mean, the horizontal line inside each box represents the median, the bottom and top of each box represent the 25th and 75th percentiles, and the whiskers represent the range of the data excluding outliers. FIG. 8B shows a similar figure for time in range 70-180 mg/dL. Time in range 70-180 mg/dL was calculated for each participant by pooling all data prior to the 5-year visit.

FIG. 9 is Table 1 entitled GLYCATED HEMOGLOBIN AND VIRTUAL CGM METRICS BY TREATMENT GROUP. Footnote a shows Mean glycated hemoglobin was calculated for each participant by taking a weighted average of all follow-up measurements. Each measurement was weighted by the time since the last collection date to account for the fact that the timing of glycated hemoglobin measurements varied across participants. All variables were analyzed as continuous variables. Footnote b shows Virtual CGM metrics that were calculated by pooling data over the entire follow-up period. Footnote c shows that the HBGI is computed using a risk transformation of the blood glucose measurements scale. The formula can be found in various publications, including two of the papers cited here. [5,18] CGM, continuous glucose monitoring; CI, confidence interval; SD, standard deviation.

FIG. 10 is Table 2 entitled HAZARD RATIOS FOR THE DEVELOPMENT OR PROGRESSION OF RETINOPATHY. Footnote a shows hazard ratios were derived from discrete Cox proportional hazard models with the glycemic metric included as a time-dependent covariate calculated by pooling all data prior to the event date. The adjusted models were stratified by early treatment diabetes retinopathy study (ETDRS) TDRS score at baseline and adjusted for age, sex, and duration of diabetes separately for the primary and secondary cohorts. The hazard ratios were calculated using the same values of change, for example, 10% for TIR, 15 mg/dL for average glucose, which were defined in a previous publication validating TIR as outcome measure. [13] Footnote b shows that the HBGI is computed using a risk transformation of the blood glucose measurements scale. The formula can be found in various publications, including two of the papers cited here. [5,18] TIR, time-in-range.

FIG. 11 is Table 3 entitled HAZARD RATIOS FOR THE DEVELOPMENT OF MICROALBUMINURIA shows at Footnote a that hazard ratios were derived from discrete Cox proportional hazard models with the glycemic metric included as a time-dependent covariate calculated by pooling all data prior to the event date. The adjusted models were stratified by ETDRS score at baseline and adjusted for age, sex, and duration of diabetes separately for the primary and secondary cohorts. The hazard ratios were calculated using the same values of change, for example, 10% for TIR, 15 mg/dL for average glucose, which were defined in a previous publication validating TIR as outcome measure. [13] Footnote b shows that the HBGI is computed using a risk transformation of the blood glucose measurements scale. The formula can be found in various publications, including two of the papers cited here. [5,18] with HR meaning hazard ratio.

FIG. 12 is Supplementary Table S1 entitled Glycated Hemoglobin and Virtual CGM Metrics according to Development or Progression of Retinopathy. The outcome, development or progression of retinopathy, is defined as the progression of three or more steps from baseline on the interim ETDRS scale at two consecutive follow-up visits 6 months apart. Mean glycated hemoglobin was calculated for each participant by taking a weighted average of all follow-up measurements. Each measurement was weighted by the time since the last collection date to account for the fact that the timing of glycated hemoglobin measurements varied across participants. Virtual CGM metrics were calculated by pooling data over the entire follow-up period. The HBGI is computed using a risk transformation of the blood glucose measurements scale. The formula can be found in various publications, including a paper cited here. [7]

FIG. 13 is Supplementary Table S2 entitled Glycated Hemoglobin and Virtual CGM Metrics according to Development of Microalbuminuria. The outcome, development of microalbuminuria, was defined as an albumin excretion rate (AER)≥30 mg/24 h at two consecutive follow-up visits 12 months apart. The analysis cohort for this outcome consisted of all participants with an AER<30 mg/24 h at baseline and at least one follow-up AER measurement. Mean glycated hemoglobin was calculated for each participant by taking a weighted average of all follow-up measurements. Each measurement was weighted by the time since the last collection date to account for the fact that the timing of glycated hemoglobin measurements varied across participants. Virtual CGM metrics were calculated by pooling data over the entire follow-up period. The HBGI is computed using a risk transformation of the blood glucose measurements scale. The formula can be found in various publications, including a paper cited here. [7]

FIG. 14 is Supplementary Table S3 entitled Glycated Hemoglobin and Virtual CGM Metrics according to Development of Neuropathy at 5 Years. The outcome, development of neuropathy, was defined as a positive finding or neuropathy at 5 years based on nerve conduction tests. The analysis cohort for this outcome consisted of participants with no neuropathy at baseline and a non-missing neuropathy assessment at 5 years. Mean glycated hemoglobin was calculated for each participant by taking a weighted average of all follow-up measurements up to 5 years. Each measurement was weighted by the time since the last collection date to account for the fact that the timing of glycated hemoglobin measurements varied across participants. Virtual CGM metrics were calculated by pooling data over the 5-year follow-up period. The HBGI is computed using a risk transformation of the blood glucose measurements scale. The formula can be found in various publications, including a paper cited here. [7]

FIG. 15 is Supplementary Table S4 entitled Odds Ratios for the Development of Neuropathy at 5 Years. Odds ratios were derived from logistic regression models with the glycemic metric included as a covariate calculated by pooling all data up to 5 years. The adjusted models also included covariates for ETDRS score at baseline, age, sex, and duration of diabetes separately for the primary and secondary cohorts. The hazards ratios were calculated using the same values of change, e.g., 10% for TIR, 15 mg/dL for average glucose, which were defined in a previous publication validating TIR as outcome measure. [8] The HBGI is computed using a risk transformation of the blood glucose measurements scale. The formula can be found in various publications, including a paper cited here. [7]

FIG. 16 is a computer environment that may be used in accordance with this disclosure.

### DETAILED DESCRIPTION

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the disclosed technology. Similarly, it is also to be understood that the mention of one or more components in a device or system does not preclude the presence of additional components or intervening components between those components expressly identified.

As discussed herein, a “subject” (or “patient”) may be any applicable human, animal, or other organism, living or dead, or other biological or molecular structure or chemical environment, and may relate to particular components of the subject, for instance specific organs, tissues, or fluids of a subject, may be in a particular location of the subject, referred to herein as an “area of interest” or a “region of interest.”

A detailed description of aspects of the disclosed technology, in accordance with various example embodiments, will now be provided with reference to the accompanying drawings. The drawings form a part hereof and show, by way of illustration, specific embodiments and examples. In referring to the drawings, like numerals represent like elements throughout the several figures. An aspect of an embodiment of the present disclosure provides, among other things, a system, method and computer readable medium for providing a computer implemented paradigm that leverages the power of reinforcement learning (RL) to address challenges in insulin control for diabetes.

Numerous studies primarily cross-sectional, have demonstrated a strong association between TIR, measured with CGM, and diabetic complications.12 None of these studies, however, had the breadth of the DCCT with respect to the methods of classification of diabetic complications and the long duration of follow-up. Thus, in aggregate, these studies have not achieved the adoption of CGM-based metrics by regulatory agencies. One study utilized the DCCT dataset to derive a TIR measure from the DCCT's collection of blood glucose (BG) samples at seven time points during one day every 3 months. Despite the limited number of data points, a strong association between this derived TIR metric and vascular complications was found.13 This article introduces a radically new idea: use of contemporary data science methods to reproduce virtual CGM traces for the DCCT participants, thereby upsampling the original sparse DCCT BG data. To do so, we developed a multistep machine-learning procedure, which was trained on archival self-monitored blood glucose (SMBG) data, and then sequentially added CGM traces filling in the monthlong, or 3-month-long, gaps in the original data, for each of the 1441 DCCT participants over the 10 years of study.

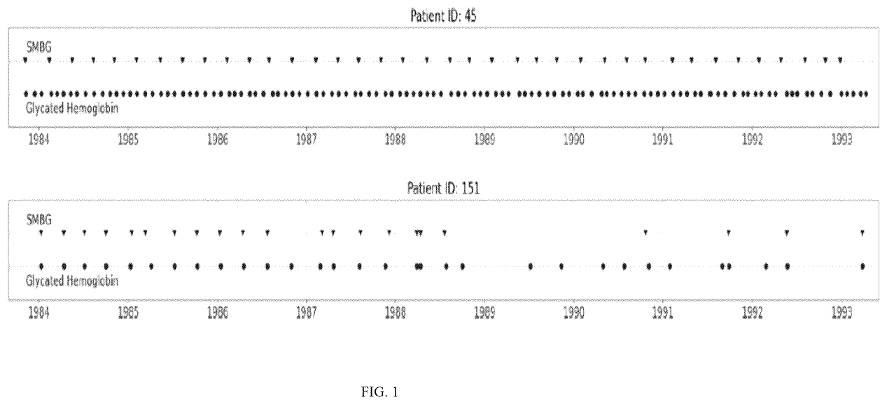

This article then utilized these virtual CGM profiles to compute TIR and other CGM metrics and evaluate the predictive ability of these metrics for the development or worsening of retinopathy, nephropathy, and neuropathy in the DCCT dataset. Research Design and Methods The DCCT followed 1441 participants with type 1 diabetes, randomized to intensive treatment (n=711) versus conventional therapy (n=730) for up to 10 years.1 The original DCCT data were obtained from the National Institute of Diabetes and Digestive and Kidney Diseases (NIDDK). Glycemic control data included glycated hemoglobin determinations every month (intensive group) or every 3 months (conventional treatment group) and 7-point capillary BG profiles collected during one day every 3 months in both groups. FIG. 1 illustrates typical data collected by the DCCT participants: upper panel—a participant in the intensive treatment group (Patient ID: 45); lower panel—a participant in the conventional treatment group (Patient ID: 151). Diabetes complications data included the development of or worsening of retinopathy as determined from retinal photographs by a central reading center; development of nephropathy (microalbuminuria), defined as albumin excretion>30 mg per 24 h; and neuropathy, defined as an abnormal neurological examination consistent with the presence of peripheral sensorimotor neuropathy, plus either abnormal nerve conduction in at least two peripheral nerves or unequivocally abnormal autonomic nerve testing.1 To design the data up sampling procedure, the University of Virginia Center for Diabetes Technology initially utilized glycated hemoglobin and 7-point capillary blood profiles from 20% of DCCT patients. A multistep machine-learning procedure was devised, composed of three sequentially acting algorithms:

Algorithm 1 was trained on 3-month segments of archival SMBG traces14 to model intra-and interday BG variability by creating appropriate distributions of SMBG data. Further, each 3-month SMBG segment was also associated with a corresponding value of glycated hemoglobin estimated from SMBG data using a previously introduced model of hemoglobin glycation and clearance with fixed population parameters. Algorithm 2 used the SMBG traces generated by Algorithm 1 and ran across the 7-point daily capillary BG profiles and glycated hemoglobin values of each DCCT participant to associate each 7-point daily profile with a 3-month SMBG segment. The SMBG segment selection was done by maximizing the likelihood that a profile was sampled from the distributions associated with the segment and minimizing the distance between glycated hemoglobin values.

Algorithm 3 used a previously defined and validated finite set of CGM motifs,15-17 to add CGM traces to SMBG segments for each DCCT participant. We should underscore that all used SMBG readings and CGM data were real and were assigned to DCCT participants when the procedure learned their individual glycemic characteristics and found similar traces in the archival databases. A detailed description of this procedure and a link to the software code used to compute the three algorithms are given in the Supplementary Appendix. When the procedure was completed and fixed, the procedure was applied to all DCCT data, and the complete virtual CGM traces were provided to the JAEB Center for Health Research for analysis of the relationship between CGM metrics and complications. First, a validation of key results was performed against the original DCCT article,1 aiming to establish that the data were retrieved correctly from the 30-year-old archives. Statistical analyses were carried out according to the Statistical Analysis Plan. Commonly accepted metrics were calculated from the virtual CGM data and then evaluated as potential predictors of diabetes complications: TIR 70-180 mg/dL, time in tight range 70-140 mg/dL, mean glucose, time>140 mg/dL, time>180 mg/dL, time>250 mg/dL, and high BG index.5,6 Poisson regressions were used to assess the association of these metrics with diabetes complications. Sensitivity analysis was performed to explore if CGM metrics calculated over different intervals (e.g., 14, 30, or 90 days prior to each DCCT glycated hemoglobin collection) produced similar results. For all analyses, confidence intervals are reported at the 95% level and P-values are assessed at the a=0.05 significance level. Results Validation of the data retrieval Supplementary Figure S1 presents the glycated hemoglobin values observed in intensive vs. conventional treatment groups of the DCCT (Panel A) and the same results reproduced 30 years later (Panel B).

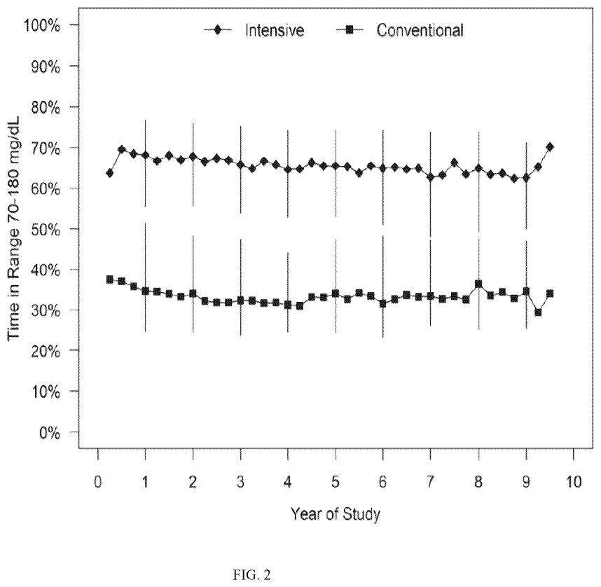

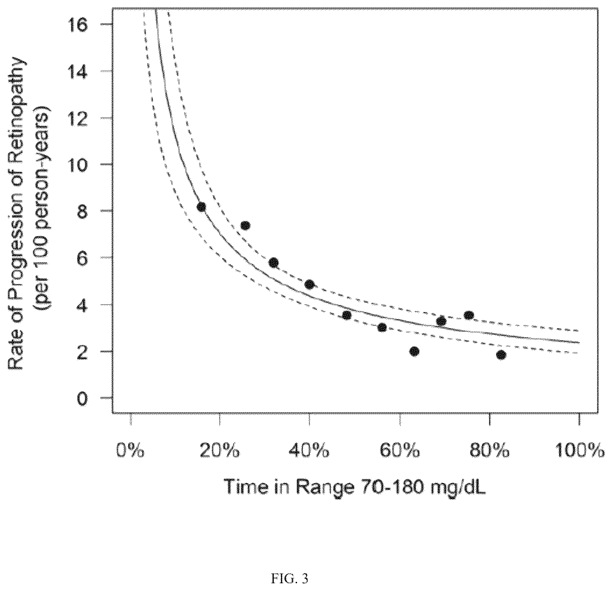

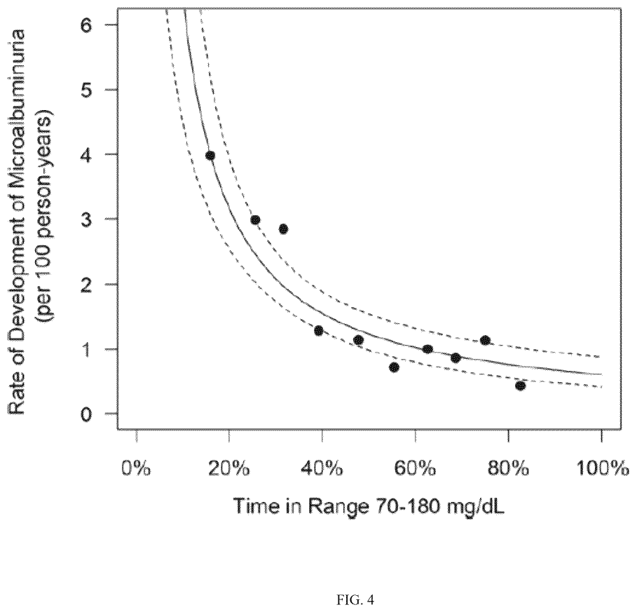

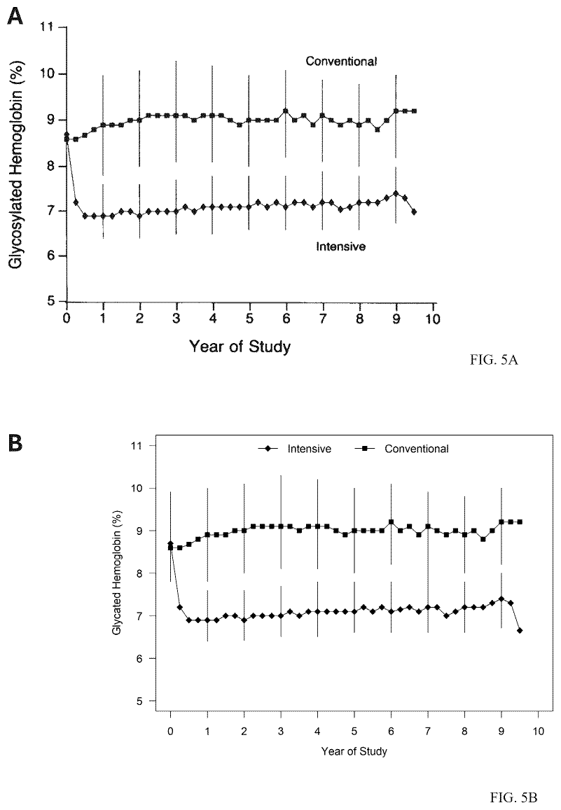

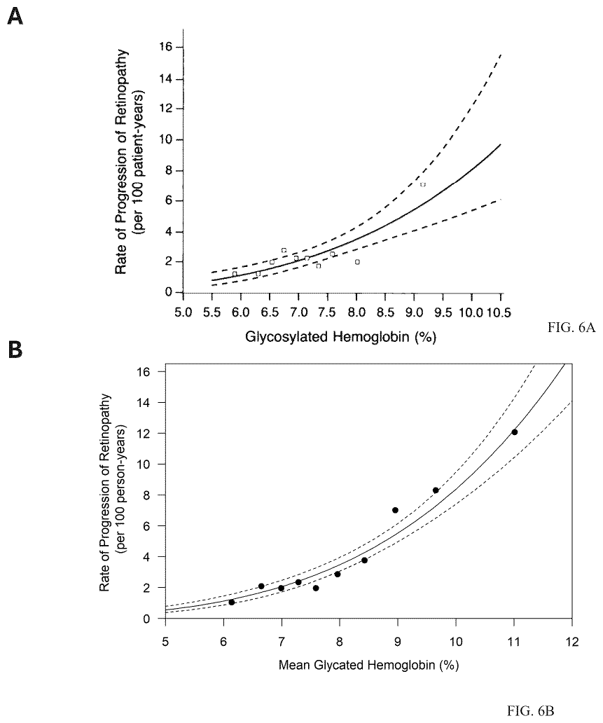

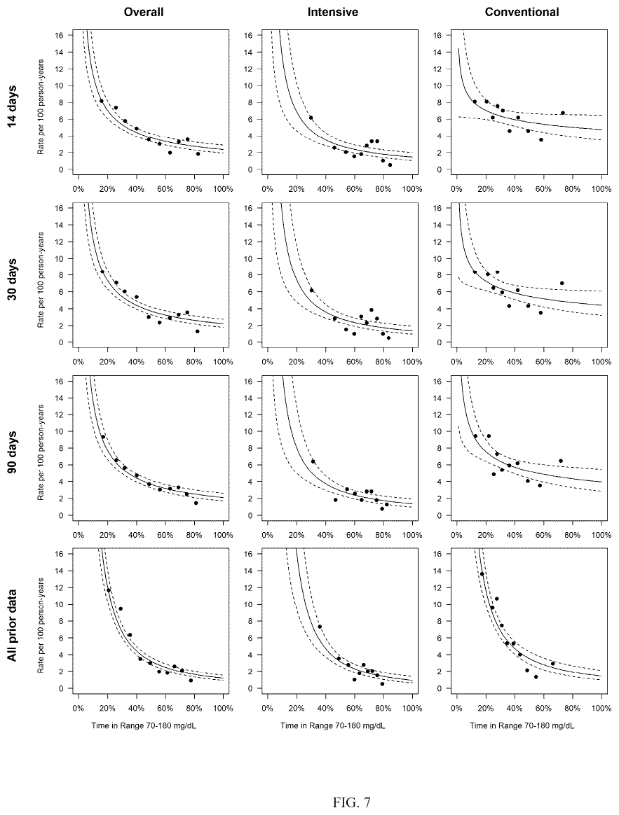

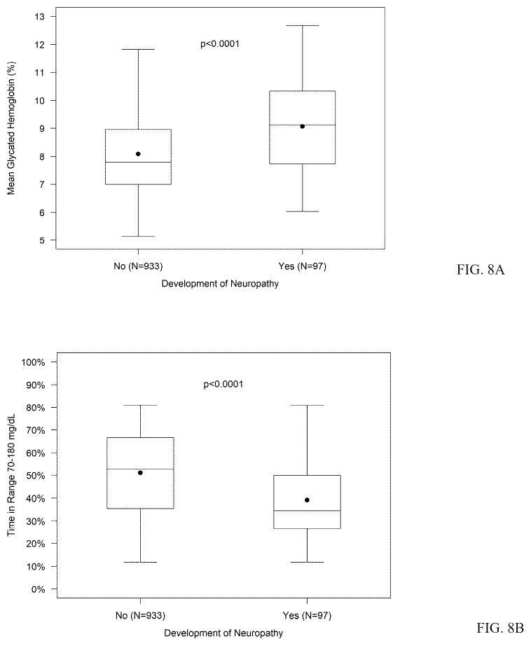

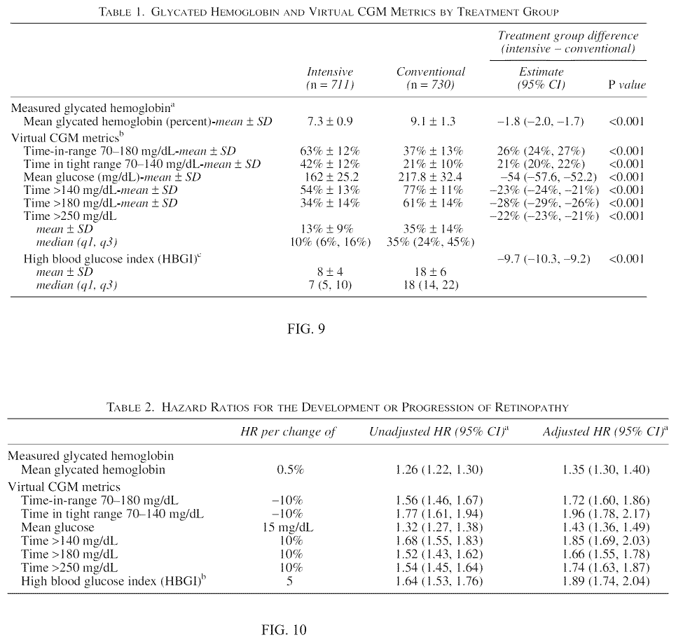

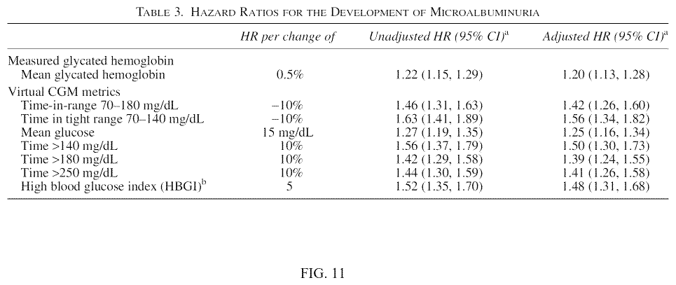

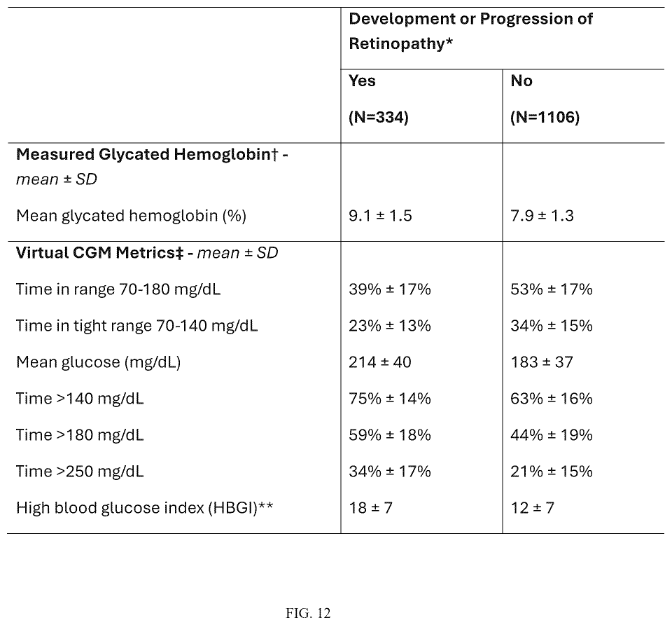

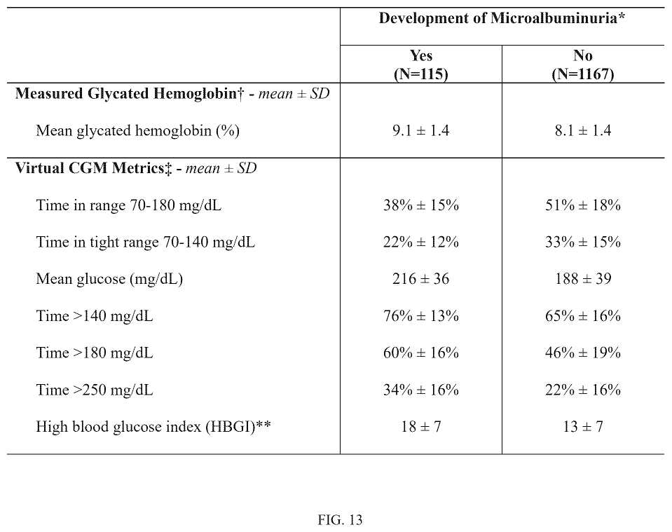

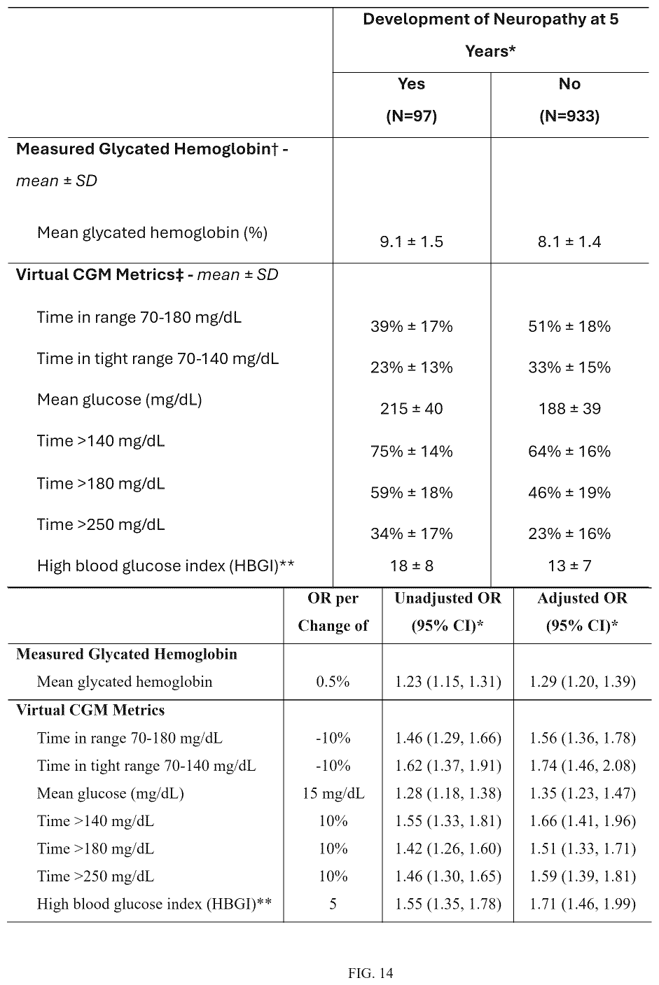

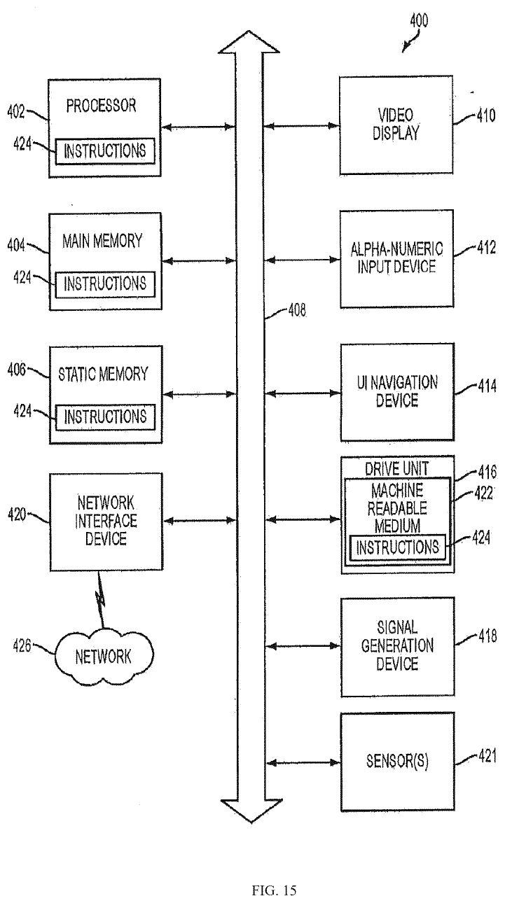

Supplementary Figure S2 presents the relationship between glycated hemoglobin and rate of progression to retinopathy per 100 patient years in the original intensive treatment DCCT group (Panel A) and as reproduced now for all DCCT participants (Panel B). Here we chose to reproduce this relationship for the entire DCCT cohort for two reasons: (i) the amplitude of retinopathy for all participants is greater than the intensive group alone, and (ii) this figure is used later in the results for comparison with TIR. However, if Panel B is limited to the intensive treatment group, the original DCCT figure is reproduced. Intensive versus conventional treatment The time course of TIR over the 10 years of DCCT is presented in FIG. 2. For this figure, TIR was computed using 14 days of virtual CGM data prior to each glycated hemoglobin determination. It is evident that the time course of TIR mirrors the time course of glycated hemoglobin observed in the DCCT (Supplementary Fig. S1). Table 1 includes glycated hemoglobin and all CGM-based metrics stratified by treatment group, together with estimated 95% confidence intervals and significance levels. The differences between intensive versus conventional treatment are highly significant, P<0.001 for all metrics, and demonstrate the consistency between glycated hemoglobin and CGMbased metrics. Retinopathy The relationship of retinopathy with 14-day TIR is presented in FIG. 3 for the entire DCCT population. Compared to Supplementary Figure S2, Panel B, the relationship between TIR and the rate of retinopathy per 100 patient years is virtually identical, but inverse, to the relationship between glycated hemoglobin and retinopathy. Lines represent the estimate and 95% confidence interval from a Poisson regression model for rate of retinopathy. Supplementary Figure S3 presents the sensitivity of the relationship of rate of retinopathy with TIR, to number of days of CGM data collection, overall and for each treatment group. It is evident that for the entire DCCT population, CGM data assessed 14, 30, 90, or all available days prior to each glycated hemoglobin determination produce very similar results. When DCCT participants were separated by treatment group, TIR based on all prior data produced slightly better results than the limited-time CGM traces. Supplementary Table S1 presents glycated hemoglobin and all virtual CGM metrics according to the development or progression of retinopathy. Table 2 presents the hazard ratios for the development or progression of retinopathy according to glycated hemoglobin and CGM metrics of hyperglycemia. For most metrics, a 10 percentage points increase indicates a higher hazard increase than 0.5% increase in glycated hemoglobin, with time in tight range (70-140 mg/dL) consistently yielding highest risk increase. Microalbuminuria and neuropathy As presented in FIG. 4, the development of microalbuminuria per 100 patient years was related to TIR computed from CGM data during the 14 days prior to glycated hemoglobin determination for the entire DCCT population.

Supplementary Table S2 stratified glycated hemoglobin and all virtual CGM metrics according to the development of microalbuminuria, and Table 3 includes the corresponding hazard ratios. Similar to the results for retinopathy, the hazard ratios were higher for a 10% increase in a CGM metric than a 0.5% increase in glycated hemoglobin and are highest for time in tight range (TITR, 70-140 mg/dL). Supplementary Figure S4 presents the distributions of glycated hemoglobin (Panel A, included here because it was not presented by the original DCCT article) and TIR (Panel B) versus confirmed neuropathy at 5 years (No, Yes). For both markers, the differences were significant (P<0.001). Neuropathy was more prevalent for those with glycated hemoglobin values above 8.5% and TIR below 50%. Supplementary Table S3 presents glycated hemoglobin and virtual CGM metrics according to the development of neuropathy at 5 years. Supplementary Table S4 includes the odds ratios for the development of neuropathy at 5 years. Discussion The DCCT trial collected large amounts of data regarding the effects of intensive treatment of type 1 diabetes on diabetes complications over a long period of time, for example, 10 years.1 The DCCT glucose data, however, were sparse and limited to monthly glycated hemoglobin determinations in the intensive treatment group, quarterly in the control group, and quarterly 7-point capillary glucose profiles in both groups. Thirty years after the publication of this landmark trial, we saw an opportunity to augment the DCCT with contemporary CGM traces, that is, virtualize the trial and add CGM data to all of its 1441 participants for the 10-year duration of the study. To the best of our knowledge, reproduction, and upsampling of clinical trial data using contemporary data science and computer simulation methods have not been done to date. This is now possible in type 1 diabetes for two reasons: (1) Diabetes mellitus is one of the best quantified human conditions: in the past 40 years metabolic monitoring technologies have progressed from occasional assessment of average glycemia via glycated hemoglobin, through blood GCM a few times a day, to CGM producing data points every few minutes, that is, time series tracking the dynamics of the metabolic system. In parallel, elaborate compartmental models of glucose-insulin dynamics have been developed and are now in use for a variety of purposes, including for in silico testing of automated insulin delivery algorithms, which is recognized by the FDA as a substitute for animal preclinical trials,19,20 and (2) we have shown15 and extensively validated16 that the multitude of daily CGM profiles can be approximated by a finite set of 483 motifs, which approximate well any CGM trace observed in vivo. Because the number of distinct CGM daily profiles is finite, a machinelearning procedure can be trained to assign a motif to each day of each person's CGM trace and can then extrapolate this approximation over multiple days, as long as it learns the characteristics of this person's glycemic variability over time.18 This first-in-class experiment aimed to reproduce the DCCT data and augment the trial with CGM traces tailored to each participant's glycemic characteristics. First, we validated that the archival DCCT data were retrieved accurately 30 years after the conclusion of the trial (Supplementary Figs. S1 and Figs. S2). Then, the procedure was trained on data from 20% of DCCT patients and all elements of the procedure were fixed. Thereafter, the complete DCCT dataset was reproduced and augmented with CGM data; established CGM-based metrics5-8 were computed and used to re-analyze the DCCT microvascular complication outcomes as functions of CGM-based TIR and other characteristics related to hyperglycemia. We have demonstrated a strong association of TIR with the risk of development or progression of retinopathy and with the development of microalbuminuria. Similar strong associations were observed for other virtual CGM metrics: mean glucose; time above 140, 180, and 250 mg/dL; and time in tight range 70-140 mg/dL. As with the DCCT glycated hemoglobin findings, the risk of complications increased exponentially at higher levels of dysglycemia. Our findings are similar to those reported for the association of TIR derived from quarterly 7-point BG measurements on one day.13 However, they are far more robust by creating a CGM dataset with 288 glucose values per day for various periods prior to each quarterly glycated hemoglobin measurement, for example, 14, 30, and 90 days, or the entire CGM time series prior to glycated hemoglobin determination. Sensitivity analysis showed that 14 days of CGM, that is, a standard ambulatory glucose profile,7,21 appeared sufficient to capture the progression of microvascular complications with relevant clinical implications. We should acknowledge, however, that the method proposed here has limitations: not every individual reconstructed CGM data point should be considered accurate—such a data granularity is likely unachievable with sparse data as those collected by the DCCT. However, summary characteristics, for example, CGM metrics computed over 2 weeks or longer are accurate and offer a realistic approximation of the progression of glycemic control during the DCCT. Conducting a new DCCT to demonstrate that improvement in TIR and other CGM metrics with intensive glycemic therapy results in a reduction in chronic vascular complication rates would be cost-prohibitive and lack feasibility, also from ethical considerations. While the clinical community has long been a proponent of CGM metrics for assessing glycemic control,6-8 regulators have hesitated from accepting these glycemic measures as clinical trial endpoints. Recreating the DCCT and adding virtual CGM data to this trial provide a compelling case for CGM-measured TIR to be an acceptable endpoint for clinical trials. Given that the entire DCCT population represents well the heterogeneity of type 1 diabetes, we suggest that a 14-day CGM data collection repeated over time (e.g., every 3 months) is comparable to glycated hemoglobin in terms of assessment of retinopathy, nephropathy, and neuropathy. Looking at the near future, new technologies, such as machine learning, generative artificial intelligence, and in silico modeling, are gaining acceptance in the diabetes research community,22 and will likely lead to a variety of applications, such as the enhancement of the data of a classical clinical trial proposed here. Other possibilities include, but are not limited to: in silico drug design and optimization of medication dosing; replay simulation of clinical data testing alternative treatment strategies—for example, the treatment of the DCCT experimental group can be “replayed” using contemporary automated insulin delivery system to assess its effect on the frequency of severe hypoglycemia observed in the DCCT23; or, machine-learning methods can be used to power automated insulin delivery and thereby enhance their efficacy with the knowledge accumulated by countless in silico experiments, clinical trials, and real-life data.24 All these could in turn reduce the current burden of classical clinical trials for participants and clinical teams.

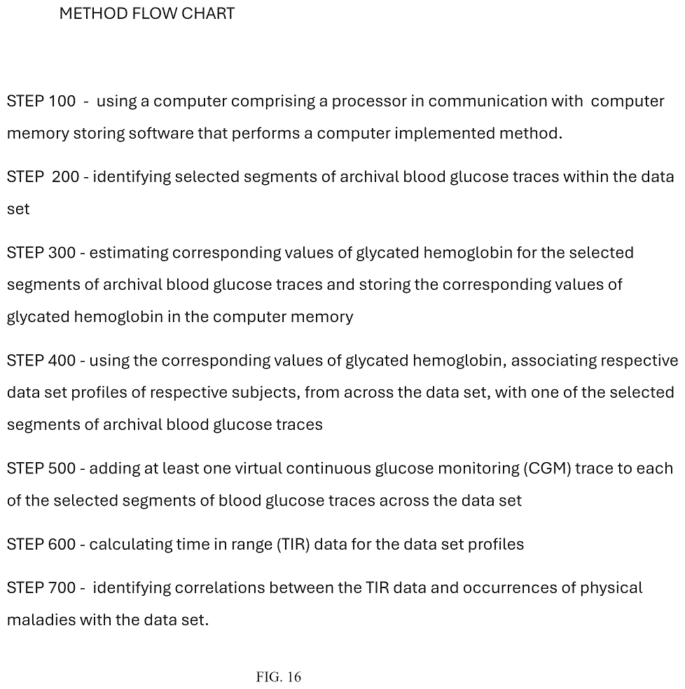

FIG. 16 is a block diagram illustrating an example of a machine upon which one or more aspects of embodiments of the present disclosure can be implemented.

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs).) Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine-readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein. Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 410, input device 412 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine-readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

### Algorithmic Procedure Description

The DCCT data [S1] up sampling procedure is a multi-step machine learning procedure composed of three sequentially acting algorithms: Algorithm 1 was trained on archival 9-month self-monitored blood glucose traces [s2] to “learn” intra-day and inter-day blood glucose variability and create appropriate distributions. Algorithm 2 ran across the 7-point daily capillary blood glucose profiles of each DCCT participant to create sequences of episodic blood glucose measurements, using in parallel a previously introduced model of hemoglobin glycation and clearance with fixed population parameters [S3] to approximate each participant's glycated hemoglobin values measured in the DCCT. Algorithm 3 used a previously validated and fixed finite set of CGM motifs [S4-S6] to add CGM traces for each DCCT participant:

### Algorithm 1—Assessing the Inter-Day Variability of Self-Monitoring Blood Glucose (SMBG) Profiles:

Archival data for 425 individuals with type 1 diabetes, each using SMBG for 9 months [S2] were divided into 3-month SMBG segments, generating a total of 29,700 available segments. For each segment, SMBG measurements were split into 7 time-of-day intervals: Early Morning: [5:00 to 9:00 h]; Late Morning: [6:30 to 10:30 h]; Midday: [10:00 to 14:00 h]; Early Afternoon: [11:30 to 15:30 h]; Late Afternoon: [16:00 to 20:00 h]; Evening: [17:30 to 21.30 h], and Night: [20:00 to 5:00 h]. For a three-month segment to be included in the analysis, each time-of-day interval was required to contain at least 30 SMBG. A lognormal probability density function (PDF) was fitted to the SMBG readings, representing the expected distribution of SMBG values in each time-of-day intervals, as follows: p_(t=k) (x μ,σ)=1/(xσ√2π) e\hat{ }((−(ln x−μ)\hat{ }2/(2σ\hat{ }2))), where: p_(t=k) is the PDF for time-of-day interval k; x is the SMBG reading; μ and σ are the parameters of the log-normal distribution, representing the mean and standard deviation of the logarithm of the SMBG readings, respectively. These parameters were estimated using maximum likelihood.

Algorithm 2—Pairing SMBG Profiles with Glycated Hemoglobin Determinations:

One hundred sixteen individuals in the Archival data had glycated hemoglobin assessed at baseline and then quarterly. [S2] The relationship between SMBG and glycated hemoglobin was described using a previously introduced dynamical model of hemoglobin glycation and clearance, driven by daily average blood glucose levels. [S3] The model operates using the following recursive formula where: eA1c(t)=0.9512×eA1c(t−1)+0.0488×f(□SMBG□_t) is the estimated glycated hemoglobin at day t;
f(SMBG)_t) is the model forcing function calculated from past SMBG readings as:
f(SMBG)_t )=4.15+0.35×mp0 is the average of SMBG measurements from the past six days.

Importantly for this investigation, the parameters were fixed at population values and values determined from the Archival data, respectively. There was no further parameter re-estimation optimizing the SMBG-glycated hemoglobin relationship for the DCCT data. Using this model, an eA1c value was associated to each 3-month SMBG segment defined in Algorithm 1.

To select the most suitable Archival data segment representing the data from each DCCT visit, scoring functions for both glycated hemoglobin and SMBG values were defined as follows: Scores were then used to identify the optimal Archival data segment for each DCCT visit, using the following two objectives:

Objective 1: select all segments where Glycated hemoglobin score is ≤0.3²; if there is no segment within the defined distance, the distance is increased by m=0.1², until at least one segment is found within the defined distance;

Objective 2: among the selected segments, choose the one with the highest SMBG score, ensuring optimal alignment with the DCCT SMBG profile.

Finally, by sampling values from the log-normal PDFs associated with the selected optimal segment, daily 7-point SMBG profiles were generated for each day of each DCCT participant. This completed the up sampling of capillary glucose values from quarterly to daily capillary blood profiles.

### Algorithm 3—Adding Continuous Glucose Monitoring (CGM) Data:

A daily CGM profile is defined as the CGM time series data from a single day (midnight to midnight), and assuming a 5-minute sampling resolution of the CGM sensor, has 288 data points. As previously defined,⁴ two daily CGM profiles match each other well if their shapes match and their relative locations in blood glucose space match. Root mean squared error (RMSE) was used as the measure of similarity between two daily CGM profiles. This measure normalizes the measure by incorporating the number of data points used in the computation, allowing for missing data in the two daily CGM profiles and making the method more general. The matching (computation of RMSE) was performed after transforming the daily CGM profiles into risk space,⁷ where the transformation ensured clinical sensitivity over the entire glucose space by enhancing the clinical resolution of the measure during hypoglycemia. Applying the matching procedure recursively resulted in a set of 483 motifs, which were representative of the variability of CGM profiles,⁴ such that almost any daily CGM profile can be approximated by a motif.

The set of 483 motifs was fixed thereafter and externally validated by using it to classify an additional 137,030 daily CGM profiles from individuals with type 1 diabetes and three different treatment modalities (multiple daily injections, insulin pump, and automated insulin delivery).⁵ This external validation set differed from the primary set in terms of demographics (data from younger individuals) and baseline characteristics (differences in the distribution of blood glucose values due to the focuses of the studies). In addition, the external validation set had temporal and geographic transportability: the studies span a 14-year time period starting in 2006, and were carried out at 43 research centers across North America.⁵ The classified daily CGM profiles were placed in a database so that, given a specific motif index, all examples of daily CGM profiles classified as the motif with that motif index can be efficiently identified.

In this study, Algorithm 3 assumed that for each day of each DCCT participant, there is a capillary blood glucose profile consisting of 4 or more readings. These data points were generated by Algorithm 2 as described above. To generate a daily CGM profile for that day we:
1. Compute the least squares estimate (score) between the capillary blood profile and each of the 483 motifs.
2. Rank the motifs by score and select those motifs where the score is within 1.05 of the minimum score taken over all 483 motifs.
3. Randomly select, for each of the selected motifs, up to 5 daily CGM profiles from the database which are classified as the motif.
4. Compute the least squares estimate (score) between the capillary blood data points and each of the randomly selected daily CGM profiles.
5. Select the daily CGM profile with the smallest score as the daily CGM profile for the day.

This completed the upsampling of the DCCT data to daily CGM profiles.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

This disclosure represents a significant advancement in skin assessment technology, utilizing state-of-the-art machine learning techniques to optimize related hardware. This disclosure integrates a sophisticated neural network-based agent that performs complex analysis of multi-modal time series data from sources

It should be appreciated that any element, part, section, subsection, or component described with reference to any specific embodiment above may be incorporated with, integrated into, or otherwise adapted for use with any other embodiment described herein unless specifically noted otherwise or if it should render the embodiment device non-functional. Likewise, any step described with reference to a particular method or process may be integrated, incorporated, or otherwise combined with other methods or processes described herein unless specifically stated otherwise or if it should render the embodiment method nonfunctional. Furthermore, multiple embodiment devices or embodiment methods may be combined, incorporated, or otherwise integrated into one another to construct or develop further embodiments of the disclosure described herein.

It should be appreciated that any of the components or modules referred to with regards to any of the present disclosure embodiments discussed herein, may be integrally or separately formed with one another. Further, redundant functions or structures of the components or modules may be implemented. Moreover, the various components may be communicated locally and/or remotely with any user/clinician/patient or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the anatomical, environmental, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein. The citation and/or discussion of such references is provided merely to clarify the description of the present disclosure and is not an admission that any such reference is “prior art” to any aspects of the present disclosure described herein. In terms of notation, “[n]” corresponds to the nth reference in the list. All references cited and discussed in this specification are incorporated herein by reference in their entireties and to the same extent as if each reference was individually incorporated by reference. It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about. ” Additional descriptions of aspects of the present disclosure will now be provided with reference to the accompanying drawings. The drawings form a part hereof and show, by way of illustration, specific embodiments or examples.

### REFERENCES

1. Nathan DM, Genuth S, Lachin J, et al. Diabetes Control and Complications Trial Research Group. The effect of intensive treatment of diabetes on the development and progression of long-term complications in insulin-dependent diabetes mellitus. N Engl J Med 1993;329(14):977-986;
2. Bergenstal RM, Beck RW, Close KL, et al. Glucose Management Indicator (GMI): A new term for estimating A1C from continuous glucose monitoring. Diabetes Care 2018;41(11):2275-2280;
3. Malka R, Nathan DM, Higgins JM. Mechanistic modeling of hemoglobin glycation and red blood cell kinetics enables personalized diabetes monitoring. Sci Transl Med 2016;8(359):359ra130;
4. Bergenstal RM, Gal RL, Connor CG, et al. T1D Exchange Racial Differences Study Group. Racial differences in the relationship of glucose concentrations and hemoglobin A1c levels. Ann Intern Med 2017;167(2):95-102;
5. Kovatchev BP. Metrics for glycaemic control—from HbA1c to continuous glucose monitoring. Nat Rev Endocrinol 2017;13(7):425-436;
6. Danne T, Nimri R, Battelino T, et al. International consensus on use of continuous glucose monitoring. Diabetes Care 2017;40(12):1631-1640;
7. Battelino T, Danne T, Bergenstal RM, et al. Clinical targets for continuous glucose monitoring data interpretation: Recommendations from the international consensus on time in range. Diabetes Care 2019;42(8):1593-1603;
8. Battelino T, Alexander CM, Amiel SA, et al. Continuous glucose monitoring and metrics for clinical trials: An international consensus statement. Lancet Diabetes Endocrinol 2023;11(1):42-57;
9. Beck RW, Bergenstal RM, Cheng P, et al. The relationships between time in range, hyperglycemia metrics, and HbA1c. J Diabetes Sci Technol 2019;13(4):614-626;
10. Fabris C, Heinemann L, Beck R, et al. Estimation of hemoglobin A1c from continuous glucose monitoring data in individuals with type 1 diabetes: Is time in range all we need? Diabetes Technol Ther 2020;22(7):501-508;
11. Diabetes Mellitus: Efficacy Endpoints for Clinical Trials Investigating Antidiabetic Drugs and Biologic Products. Guidance for Industry. Diabetes Mellitus: Efficacy Endpoints for Clinical Trials Investigating Antidiabetic Drugs and Biological Products|FDA; 2023.
12. Beck RW. The association of time in range and diabetic complications: The evidence is strong. Diabetes Technol Ther 2023;25(6):375-377;
13. Beck RW, Bergenstal RM, Riddlesworth TD, et al. Validation of time in range as an outcome measure for Diabetes Clinical Trials. Diabetes Care 2019;42(3):400-405;
14. Kovatchev BP, Mendosa P, Anderson S, et al. Effect of automated bio-behavioral feedback on the control of type 1 diabetes. Diabetes Care 2011;34(2):302-307;
15. Lobo B, Farhy L, Shafiei M, et al. A data-driven approach to classifying daily Continuous Glucose Monitoring (CGM) time series. IEEE Trans Biomed Eng 2022;69(2):654-665;
16. Lobo BJ, Kovatchev BP. External validation of a classifier of daily Continuous Glucose Monitoring (CGM) profiles. Comput Biol Med 2022;143:105293;
17. Kovatchev B, Lobo B. Clinically similar clusters of daily continuous glucose monitoring profiles: Tracking the progression of glycemic control over time. Diabetes Technol Ther 2023;25(8):519-528;
18. Kovatchev BP, Cobelli C. Glucose variability: Timing, risk analysis, and relationship to hypoglycemia in diabetes. Diabetes Care 2016;39(4):502-510;
19. Kovatchev BP, Breton MD, Man D, et al. In silico preclinical trials: A proof of concept in closed-loop control of type 1 diabetes. J Diabetes Sci Technol 2009;3(1):44-55;
20. Cobelli C, Kovatchev B. Developing the UVA/Padova type 1 diabetes simulator: Modeling, validation, refinements, and utility. J Diabetes Sci Technol 2023;17(6):1493-1505;
21. Bergenstal RM, Ahmann AJ, Bailey T, et al. Recommendations for standardizing glucose reporting and analysis to optimize clinical decision making in diabetes: The ambulatory glucose profile. J Diabetes Sci Technol 2013;7(2):562-578;
22. Jacobs PG, Herrero P, Facchinetti A, et al. Artificial intelligence and machine learning for improving glycemic control in diabetes: Best practices, pitfalls and opportunities. IEEE Rev Biomed Eng 2024;17:19-41;
23. Hypoglycemia in the Diabetes Control and Complications Trial. The Diabetes Control and Complications Trial Research Group. Diabetes 1997;46(2):271-286.
24. Kovatchev B, Castillo A, Pryor E, et al. Neural-net Artificial pancreas: A randomized crossover trial of a first-in-class automated insulin delivery algorithm. Diabetes Technol Ther 2024;26(6):375-382;
S1. Diabetes Control and Complications Trial Research Group; Nathan DM, Genuth S, Lachin J, et al. The effect of intensive treatment of diabetes on the development and progression of long-term complications in insulin-dependent diabetes mellitus. N Engl J Med. 1993;329(14):977-86. doi: 10.1056/NEJM199309303291401. PMID: 8366922.
S2. Kovatchev BP, Mendosa P, Anderson S, et al. Effect of automated bio-behavioral feedback on the control of type 1 diabetes. Diabetes Care. 2011;34(2):302-7. doi: 10.2337/dc10-1366. Epub 2011 January 7. PMID: 21216860; PMCID: PMC3024338.
S3. Fabris C, Heinemann L, Beck R, et al. B. Estimation of Hemoglobin A1c from Continuous Glucose Monitoring Data in Individuals with Type 1 Diabetes: Is Time In Range All We Need? Diabetes Technol Ther. 2020;22(7):501-508. doi: 10.1089/dia.2020.0236. PMID: 32459124; PMCID: PMC7336887.
S4. Lobo B, Farhy L, Shafiei M, et al. A Data-Driven Approach to Classifying Daily Continuous Glucose Monitoring (CGM) Time Series. IEEE Trans Biomed Eng 2022;69(2):654-665. doi: 10.1109/TBME.2021.3103127. Epub 2022 January 20. PMID: 34375274.
S5. Lobo BJ, Kovatchev BP. External validation of a classifier of daily continuous glucose monitoring (CGM) profiles. Comput Biol Med 2022;143:105293. doi: 10.1016/j.compbiomed.2022.105293. Epub 2022 February 12. PMID: 35182951.
S6. Kovatchev B, Lobo B. Clinically Similar Clusters of Daily Continuous Glucose Monitoring Profiles: Tracking the Progression of Glycemic Control Over Time. Diabetes Technol Ther. 2023;25(8):519-528. doi: 10.1089/dia.2023.0117. PMID: 37130300.
S7. Kovatchev BP. Metrics for glycaemic control—from HbA1c to continuous glucose monitoring. Nat Rev Endocrinol. 2017;13(7):425-436. doi: 10.1038/nrendo.2017.3. Epub 2017 March 17. PMID: 28304392.
S8. Beck, Bergenstal et al. Validation of Time in Range as an Outcome Measure for Diabetes Clinical Trials. Diabetes Care. 2019;42(3):400-405. doi: 10.2337/dc18-1444. PMID: 30352896; PMCID: PMC6905478.

1. A computer implemented method of using blood glucose control data as a medical diagnostic tool, the method comprising:
using a computer comprising a processor in communication with computer memory storing software that performs computer implemented steps comprising:
identifying selected segments of archival blood glucose traces within the data set;
estimating corresponding values of glycated hemoglobin for the selected segments of archival blood glucose traces and storing the corresponding values of glycated hemoglobin in the computer memory;
using the corresponding values of glycated hemoglobin, associating respective data set profiles of respective subjects, from across the data set, with one of the selected segments of archival blood glucose traces;
adding at least one virtual continuous glucose monitoring (CGM) trace to each of the selected segments of blood glucose traces across the data set;
calculating time in range (TIR) data for the data set profiles; and
identifying correlations between the TIR data and occurrences of physical maladies with the data set.

2. The computer implemented method of claim 1, wherein accessing selected segments of archival blood glucose traces comprises using the software to identify glycemic characteristics of blood glucose data for portions of the data set and locating similar archival blood glucose traces from the entire data set.

3. The computer implemented method of claim 1, wherein the archival blood glucose traces comprise self-monitored blood glucose (SMBG) traces, and the method further comprises using the selected segments to model intra-day and inter-day blood glucose variability by creating appropriate distributions of SMBG data.

4. The computer implemented method of claim 3, wherein associating respective data set profiles comprises maximizing a likelihood that a respective data set profile was sampled from the distributions associated with a selected segment and minimizing a distance between glycated hemoglobin values.
The computer implemented method of claim 4, wherein associating data set profiles comprises associating a plurality of 7 point daily of blood glucose profiles of the subjects within the data set with a selected segment.

6. The computer implemented method of claim 1, further comprising identifying additional correlations among the TIR data, the occurrences of physical maladies, and the archival blood glucose traces within the data set.

7. The computer implemented method of claim 6, further comprising comparing an error rate for the TIR data predicting the occurrences of the physical maladies to a respective error rate for the archival blood glucose traces predicting the occurrences of the physical maladies.

8. The computer implemented method of claim 7, wherein the software stored in computer memory comprises instructions to use the TIR data as an indicator for probability of the occurrences of physical maladies.

9. The computer implemented method of claim 8, wherein the physical maladies comprise at least one of retinopathy, nephropathy, and neuropathy.

10. The computer implemented method claim 1, further comprising dividing the data set into segments and intervals within the segments and identifying selected segments using selected blood glucose traces having intervals that that meet a threshold value for the number of blood glucose traces.

11. The computer implemented method of claim 1, wherein adding a virtual continuous glucose monitoring (CGM) trace to each of the selected segments comprises steps including:
computing a least squares estimate score between data set profiles and motifs of the virtual CGM traces;
ranking the motifs by score and selecting those motifs where the score is within a selected threshold;
randomly selecting, for each of the selected motifs, virtual CGM profiles which are classified as matching a respective motif.
computing the least squares estimate (score) between capillary blood data points and each of the randomly selected daily CGM profiles.

12. The computer implemented method of claim 11, wherein motifs of the virtual CGM traces comprise CGM profiles that meet a finite set of criteria.

13. The computer implemented method of claim 12, wherein a respective motif approximates other CGM profiles that meet the set of criteria.
